(19) United States
(12) Patent Application Publication
(10) Pub. No.: US 2010/0273908 A1
(43) Pub. Date: Oct. 28, 2010

(54) NCO PREPOLYMERS HAVING A LOW CONTENT OF FREE MONOMERIC DIISOCYANATE, AND THE PRODUCTION THEREOF

(75) Inventors: Hartmut Nefzger, Pulheim (DE); James-Michael Barnes, Breitscheid (DE); Eduard Mayer, Dormagen (DE); Joachim Wagner, Koeln (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/830,755

(22) Filed: Jul. 6, 2010

Related U.S. Application Data
(63) Continuation of application No. 12/128,124, filed on May 28, 2008, now abandoned.

(30) Foreign Application Priority Data
Jun. 1, 2007 (DE) 102007025659.2

(51) Int. Cl. C08G 18/76; C07C 263/16; C07C 265/12; C08J 9/00

(52) U.S. Cl. 521/163; 560/336; 560/360; 528/68

(57) ABSTRACT

The present invention relates to NCO prepolymers having a low content of free monomeric diisocyanate, to a process for the production of these NCO prepolymers, to polyurethanes prepared from these NCO prepolymers and to processes for the production of these polyurethanes.

### CROSS REFERENCE TO RELATED PATENT APPLICATION

The present patent application claims the right of priority under 35 U.S.C. §119 (a)-(d) of German Patent Application No. 10 2007 025 659.2 filed Jun. 1, 2007.

### BACKGROUND OF THE INVENTION

The present invention relates to NCO prepolymers which have a low content of free monomeric diisocyanate, to the production of these NCO prepolymers, to polyurethanes prepared from these NCO prepolymers and to processes for the production of such polyurethanes.

Polyurethane elastomers have been known for a long time. One of the possible production methods comprises the synthesis route via NCO prepolymers, which are reacted in a chain extension reaction with short-chained diol or short-chained diamine to form the end product. The chain extension reaction is carried out, as regards the stoichiometry, in such a manner that at least approximate equivalence of isocyanate groups on the one hand and hydroxyl or amino groups on the other hand is ensured. It is thereby possible to build up a high molecular weight.

The NCO prepolymers are obtained by reacting a long-chained polyol, in many cases a long-chained diol, either a polyether diol or polyester diol, with polyisocyanates, generally in most cases diisocyanates. In the synthesis of the NCO prepolymers, the chosen stoichiometry deviates considerably from 1:1 stoichiometry in order to prevent the build up of high molecular weights, which would lead to unmanageable viscosities of the prepolymers. Thus, for example, a 2:1 molar excess of a diisocyanate to a long-chained diol may be chosen. It is thereby ensured, on the one hand, that not all the NCO groups are able to react to completion and some NCO groups are available for the subsequent chain extension reaction, and, on the other hand, that the build up of the molecular weight remains limited and systems with still manageable viscosity are formed.

When bifunctional isocyanates and bifunctional long-chained polyols are used, however, the formation of a 2:1 adduct occurs only in the statistical mean even when a 2:1 stoichiometry is used. However, in reality, the picture is far more complicated and is readily comprehensible on the basis of statistical considerations: If a diisocyanate molecule reacts not only with one of its two NCO groups but with both, so-called pre-extension occurs, i.e. instead of a 2:1 adduct there is thereby formed a 3:2 adduct which likewise has 2 NCO end groups, that is to say is just as amenable to a subsequent chain extension reaction as a 2:1 adduct. Owing to the given total stoichiometry of 2:1, however, any pre-extension reaction also means that an unreacted diisocyanate molecule must remain in the reaction mixture for lack of a reaction partner. This is referred to as free monomeric diisocyanate.

Of course, 3:2 adducts can also be pre-extended still further, for example to form 4:3, 5:4 adducts, etc., resulting in this case too in further free monomeric diisocyanates.

In the case of diisocyanates whose NCO groups have exactly equal reactivity, the content of free monomeric diisocyanate can be calculated by means of Schulz-Flory statistics.

The probability (W) that a diisocyanate molecule does not react, that is to say remains as a free monomeric diisocyanate molecule, is given by the formula (I)

W=(1−p)² (I),

wherein p denotes the proportion of reacted NCO groups and (1−p) accordingly denotes the proportion of unreacted NCO groups and is ultimately given directly by the stoichiometry. In the case of purely bifunctional components, with a stoichiometry of 2:1, for example, only 50% of all the NCO groups are able to react, so that p, like (1−p), has the value 0.5 and W is accordingly calculated as 0.25. This means that a quarter of all the diisocyanate molecules used does not find a reaction partner and remains in the reaction product as free monomeric diisocyanate. Of course, these molar facts can easily be converted into amounts by weight in the case of polyols and polyisocyanates which are known concretely.

Diisocyanates which fulfil the requirement for equal reactivity at least very largely are, for example, 4,4′-diphenylmethane diisocyanate, hexamethylene diisocyanate and trans-cyclohexane diisocyanate.

The content of free monomeric diisocyanates in NCO prepolymers is particularly disruptive when they are readily volatile, because NCO prepolymers must in most cases be handled at elevated temperature and undesirable substances can thus be released, and this in turn has to be counteracted by complex technical protective measures, for example extraction systems.

For toluene diisocyanate (TDI), a value below 0.1 wt. % must be maintained with regard to the free monomeric diisocyanate content, at least because of legal specifications. Otherwise, the mentioned protective measures must be taken.

On account of these facts, a number of technical solutions to this problem have become known in the past.

It is therefore obvious, inter alfa, to use only those diisocyanates which have a high boiling point, i.e. low volatility. An example thereof is 4,4′-diphenylmethane diisocyanate (MDI). However, such an approach has the fundamental disadvantage that, by restricting the NCO prepolymers to those prepared from only high-boiling diisocyanates, the potential property spectrum of the resultant polyurethanes produced therefrom is not used to the full. Furthermore, MDI in particular has the disadvantage that its prepolymers have comparatively high viscosities.

It is, of course, also possible to use diisocyanates whose NCO groups have different reactivities such as, for example, 2,4-toluene diisocyanate (2,4-TDI). In such cases, the above-mentioned formula for calculating the free monomeric diisocyanate can no longer be used in pure form but must be expanded by a factor which takes account of the different reactivities. The end result of such a measure is that the pre-extension is suppressed or at least reduced, so that the proportion of free monomeric diisocyanate is consequently and in some cases markedly smaller than in the case of diisocyanates having isocyanate groups of equal reactivity.

Furthermore, from the technical point of view it is always possible to produce the prepolymer using a molar excess of diisocyanate relative to the polyol component, and to reduce the proportion of free monomeric diisocyanate to the desired value, in the case of TDI, for example, to below 0.1 wt. %, by distillation processes such as, for example, short-path evaporation or thin-layer evaporation or, for example, also by extraction processes. However, a fundamental disadvantage is the high technical outlay associated therewith. Examples of such procedures are described in, for example, DE 42 32 015 A1, DE 41 40 660 A1 and DE 37 39 261 A1.

In addition to the choice of diisocyanates having NCO groups with different reactivities and the option of subsequently removing the free monomeric diisocyanate, the product composition can, of course, also be influenced, according to the above-mentioned formula for calculating the proportion of free monomeric diisocyanate, by the parameter p, which describes the conversion of isocyanate groups. When the parameter p assumes values that are as high as possible, the proportion of free monomeric diisocyanate rapidly becomes smaller in particular as a result of the quadratic term. This means that particularly suitable stoichiometric ratios are those in which p has a value of less than 0.5, i.e. the NCO prepolymer is composed of 1 mole of diol and less than 2 moles of diisocyanate. As a result of such a measure, a NCO prepolymer that also exhibits pre-extension in the mean is of course formed. It is naturally to be expected that the viscosity will increase as a result of such a composition as compared with variants produced with a higher NCO excess.

The object of the invention was, therefore, to provide a process which allows prepolymers having a low content of free monomeric toluene diisocyanate and a low viscosity to be produced without having to carry out complex process steps, such as distillation, etc.

Surprisingly, it is possible to produce prepolymers having proportions of free monomeric TDI of less than 0.1 wt. % and viscosities, measured at 50° C., of below 5000 meas. These prepolymers can be used very successfully for a chain extension reaction with aromatic amine chain extenders and to yield polyurethane elastomers having good mechanical or mechanical-dynamic properties. In addition, the prepolymers do not have to be subjected to a process step of distillation and/or extraction.

### SUMMARY OF THE INVENTION

The invention therefore provides a process for the production of NCO prepolymers based on toluene diisocyanate having a content of free monomeric toluene diisocyanate of not more than 0.1 wt. % and a viscosity, measured at 50° C., of not more than 5000 mPas. This process requires
(1) reacting, in a one-step procedure, at reaction temperatures of from 40 to 95° C.,
   (a) one or more polyether polyols having at least 85% secondary OH groups,
   with
   (b) toluene diisocyanate having a proportion of 2,4-isomer of more than 99.5 wt. %,
   with a molar ratio of isocyanate groups to hydroxyl groups (i.e. the isocyanate index) in the range from 1.52:1 to 1.85:1 being maintained, and with the proviso that the index does not exceed the value defined by formula (I):

indexₘₐₓ=1.5982+7/hydroxyl number of the polyether

The proportion of 2,4-isomer in the toluene diisocyanate used as component (b) is preferably more than 99.6 wt. %, and more preferably more than 99.7 wt. %.

The invention further provides NCO prepolymers based on toluene diisocyanate having a content of free monomeric toluene diisocyanate of not more than 0.1 wt. % and a viscosity, measured at 50° C., of not more than 5000 mPas. These are the one-step reaction product, at reaction temperatures of from 40 to 95° C., of:
(a) one or more polyether polyols having at least 85% secondary OH groups, with
(b) toluene diisocyanate having a proportion of 2,4-isomer of more than 99.5 wt. %, in a molar ratio of isocyanate groups to hydroxyl groups (i.e. isocyanate index) in the range from 1.52:1 to 1.85:1, wherein the index does not exceed the value defined by formula (I):

indexₘₐₓ=1.5982+7/hydroxyl number (I).

The proportion of 2,4-isomer in the toluene diisocyanate is preferably more than 99.6 wt. %, and more preferably more than 99.7 wt. %.

### DETAILED DESCRIPTION OF THE INVENTION

Suitable polyether polyols for component (a) herein includes compounds that are obtained by reacting suitable starter polyols, in most cases starter diols such as, e.g. 1,2-propylene glycol, 1,4-butanediol, as well as water, by ring-opening polymerization with ethylene oxide and/or propylene oxide. It is, of course, possible to use both the base-catalyzed variant, in which strong inorganic bases, such as, for example, potassium hydroxide, are used as catalysts, and the double-metal-catalyzed variant. The polyether polyols used also include copolyethers, in which more than one aliphatic epoxide polymerizable by ring-opening polymerization is used, wherein both block-wise and random incorporation of the epoxides is possible. Suitable polyether polyols for component (a) have at least 85% secondary OH groups, based on 100% of the OH end groups. Preference is given to polyether polyols and copolyether polyols which are produced using ethylene oxide and propylene oxide and in which more than 85% of the OH end groups are secondary. Preference is given to polyether polyols which are produced using bifunctional starters, including water, and have hydroxyl functionalities of at least 1.97. Of course, mixtures of more than one polyether polyol can also be used. Preference is given to those polyether polyols which have hydroxyl numbers from 25 to 125 mg KOH/g and to mixtures whose mixed hydroxyl number is in the range from 25 to 125 mg KOH/g.

The NCO prepolymers according to the invention are obtained only when the starting toluene diisocyanate used as component (b) has a proportion of 2,4-TDI of more than 99.5 wt. %, preferably of more than 99.6 wt. %, and more preferably of more than 99.7 wt. %.

The (isocyanate) index must be maintained with particular care. The NCO prepolymers according to the present invention are obtained when the index is set as follows in dependence on the OH number of the polyether polyol, which in turn must be in the range from 25 to 125:

indexₘₐₓ=1.5982+7/hydroxyl number of the polyether

If the maximum (isocyanate) index is exceeded, NCO prepolymers that contain more than 0.1 wt % free monomeric TDI are obtained, and particularly when the prepolymer synthesis is carried out at the upper margin of or beyond the temperature window. In theory, no lower limits are set for the index. It must, however, be taken into consideration that although monomer-free NCO prepolymers are obtained as the index falls increasingly below the maximum possible index, they are no longer processable because of their greatly increased viscosity, i.e. they can no longer be stirred with a chain extender. Regardless of this, such prepolymers are still entirely usable for other applications including, for example, as moisture-curing films for knife application. In one embodiment the prepolymer is applied onto a surface, e.g. a glass sheet, for example, using a coating knife. The prepolymer can also be applied by spin coating. Said evenly spread NCO prepolymers are then cured, preferably at elevated temperatures, by the moisture in the atmosphere.

For the preparation of PUR elastomers, the NCO prepolymers are reacted with aromatic amine chain extenders, preferably using NCO prepolymers having an index which is not more than 0.1 index units below the maximum index (see equation I).

The process according to the invention is characterized in that it is carried out at elevated temperature, preferably in the temperature range from 40 to 95° C. Although low temperatures, with an otherwise identical formulation, lead to lower contents of free monomeric TDI, they also result in markedly longer reaction times, whereas higher temperatures shorten the reaction time but also allow the contents of free monomeric TDI to increase. Therefore, reaction temperatures from 55 to 90° C. are particularly preferred.

The reaction can, of course, be carried out under elevated or reduced pressure or at normal pressure. Normal pressure is preferred.

A catalyst can also be used.

The reaction is advantageously carried out with the exclusion of moisture. In a preferred embodiment, an inert protecting gas is used to cover the reaction mixture. Nitrogen is preferably used for that purpose.

The NCO prepolymers according to the invention can be used, for example, in the production of polyurethane casting elastomers, by reacting the prepolymers with one or more aromatic amine chain extenders to yield the casting elastomers. Suitable amine chain extenders include, for example, 3,5-diamino-4-chlorobenzoic acid isobutyl ester, 3,5-bis(methylthio)-2,4-diaminotoluene, 4,4′-diamino-2,2′-dichloro-5,5′-diethyldiphenylmethane, 2,4-diamino-3,5-diethyltoluene or isomers thereof, and mixtures of these amine chain extenders with each other and other amine chain extenders.

The invention is to be explained in greater detail by means of the examples described hereinbelow.

The following examples further illustrate details for the process of this invention. The invention, which is set forth in the foregoing disclosure, is not to be limited either in spirit or scope by these examples. Those skilled in the art will readily understand that known variations of the conditions of the following procedures can be used. Unless otherwise noted, all temperatures are degrees Celsius and all percentages are percentages by weight.

### EXAMPLES

### Raw Materials

Polyether 1: a polyether polyol prepared by propoxylating propylene glycol, having a functionality of about 2, a molecular weight of about 1000 and an OH number of about 112 mg KOH/g
Polyether 2: a polyether polyol prepared by propoxylaing propylene glycol, having a functionality of about 2, a molecular weight of about 2000 and an OH number of about 56 mg KOH/g
Polymeg 1000: α-hydro, ω-hydroxy-polyoxytetramethylene from Lyondell having a OH number of 112 mg KOH/g and a molecular weight of about 1000
Polymeg 2000: α-hydro, ω-hydroxy-polyoxytetramethylene from Lyondell having a OH number of 56 mg KOH/g and a molecular weight of about 3000
2,4-TDI: toluene diisocyanate having an NCO group content of about 48.3 wt. % and an isomeric purity of greater than about 99.6 wt. %
Aromatic Amine 1: 3,5-diamino-4-chlorobenzoic acid isobutyl ester, commercially available as RC crosslinker Baytec® 1604 from RheinChemie
Aromatic Amine 2: 3,5-bis(methylthio)-2,4-diaminotoluene, commercially available as Ethacure® 300 from Rheinchemie
Aromatic Amine 3: 4,4′-diamino-2,2′-dichloro-5,5′-diethyldiphenylmethane (MCDEA), commercially available as Lonzacure® MCDEA from Lonza
Aromatic Amine 4: 2,4-diamino-3,5-diethyltoluene (DETDA), commercially available as Lonzacure® DETDA 80 from Lonza
Oleic Acid: cis-octadec-9-enoic acid, CAS 112-80-1 from Aldrich, activator
Tinuvin®1375: a light-stabilizer, anti-oxidant mixture, commercially available as Tinuvin® B75 from Ciba

### Example 1

### Production of a NCO Prepolymer According to the Invention

4000 g (2 mol) of Polyether 2 were stirred in the course of one hour, under nitrogen, into 591.6 g (3.4 mol) of 2,4-TDI, heated to 77° C., in such a manner that the reaction temperature did not exceed 80° C. at any time. When the addition was complete, stirring was carried out for a further 10 hours at 80° C., and from that time onwards the NCO value was determined at 60-minute intervals. The reaction was terminated after 13 hours at the time when the NCO value determined by experiment was below the theoretical value of 2.52 wt. % NCO. The proportion of free monomeric IDI in the resultant prepolymer was determined by HPLC (high performance liquid chromatography) as 0.042 wt. %. The viscosity of the prepolymer was 1500 meas at 50° C.

### Examples 2-5

### According to the Invention

The NCO prepolymers were produced in accordance with the procedure set forth above for Example 1. The specific parameters for Examples 2-5 are set forth in Table 1.

TABLE 1
Synthesis and characterization of TDI prepolymers

| Examples | | | 1 | 2 | 3 | 4 | 5 |
|---|---|---|---|---|---|---|---|
| Polyether 2 | | | X | X | X | — | — |
| Polyether 1 | | | — | X | X | X | X |
| OH number polyol (mixture) | | [mg KOH/g] | 56 | 70 | 81 | 112 | 112 |
| Reaction temperature | | [° C.] | 80 | 75 | 75 | 70 | 75 |
| Index | | [NCO/OH] | 1.70 | 1.65 | 1.65 | 1.60 | 1.65 |
| Indexₘₐₓ according to | | [NCO/OH] | 1.72 | 1.70 | 1.68 | 1.66 | 1.66 |
| formula (I) | | | | | | | |
| NCO value | (theor.) | [wt. %] | 2.52 | 3 | 3.28 | 3.87 | 4.13 |
| | (exp.) | [wt. %] | 2.42 | 2.97 | 3.15 | 3.71 | 4.02 |
| Content of | 2,4-TDI | [%] | 0.023 | 0.008 | 0.009 | 0.001 | 0.020 |
| free TDI | 2,6-TDI | [%] | 0.019 | 0.022 | 0.022 | 0.019 | 0.031 |
| | Total | [%] | 0.042 | 0.03 | 0.031 | 0.020 | 0.051 |
| Viscosity | at 50° C. | [mPas] | 1500 | 2200 | 2700 | 4300 | 3600 |
| | at 60° C. | [mPas] | n.d.*) | 1200 | 1440 | 2100 | 1750 |

*)n.d.: not determined

The OH number of the polyether polyols used in Examples 2 and 3 was varied in the range from 56 to 112 by mixing polyether polyols 1 and 2. Varying the OH number resulted in the NCO values of the prepolymers covering a range from approximately 2.5 to approximately 4.1 wt. % NCO. The reaction temperatures and the ratio of NCO/OH groups (i.e. the isocyanate index) were adapted according to the OH number. Higher OH numbers tend to require a lower reaction temperature and a lower ratio of NCO/OH groups.

The content of free monomeric TDI in the resultant prepolymers was markedly below 0.1 wt. % in all cases.

Overall, the viscosity values of the resultant prepolymer were in a range permitting good further processability at 50 to 60° C.

Furthermore, a comparison of Examples 4 and 5 shows that a deviation from the index of 1.66 recommended by formula (I) has an effect on the viscosity of the resultant prepolymer, and thus on usability of the prepolymer. Example 5, with an index of 1.65, has a viscosity value of 3600 mPas (at 50° C.), while Example 4, with an index of 1.60, already has a viscosity of 4300 mPas (at 50° C.) with an otherwise identical formulation and despite being produced under gentle conditions (70° C. instead of 75° C.).

### Examples 6-9

### Production of NCO Prepolymers not According to the Invention

The NCO prepolymers of Comparison Examples 6-9 were produced in accordance with the procedure set forth above in Example 1. The process parameters for each of Examples 6-9 are set forth in Table 2.

TABLE 2
Synthesis and characterization of TDI prepolymers
not according to the invention

| Comparison Examples | | | 6 | 7 | 8 | 9 |
|---|---|---|---|---|---|---|
| Polyether 2 | | | X | X | X | X |
| 2,4-TDI | | [wt. %] | 99.8 | 99.8 | 99.8 | 80 |
| 2,6-TDI | | [wt. %] | 0.2 | 0.2 | 0.2 | 20 |
| OH number polyol | | [mg KOH/g] | 56 | 56 | 56 | 56 |
| (mixture) | | | | | | |
| Index | | [NCO/OH] | 1.90 | 1.80 | 1.75 | 1.80 |
| Indexₘₐₓ according to | | [NCO/OH] | 1.72 | 1.72 | 1.72 | 1.72 |
| formula (I) | | | | | | |
| Reaction temperature | | [° C.] | 60 | 80 | 80 | 80 |
| NCO value | (theor.) | [wt. %] | 3.24 | 2.90 | 2.73 | 2.90 |
| | (exp.) | [wt. %] | 3.15 | 2.88 | 2.69 | 2.82 |
| Content of | 2,4-TDI | [%] | n.d.*) | n.d.*) | n.d.*) | 0.016 |
| free TDI | 2,6-TDI | [%] | n.d.*) | n.d.*) | n.d.*) | 0.81 |
| | Total | [%] | 0.32 | 0.18 | 0.12 | 0.826 |

*)n.d.: not determined

The indices of Comparison Examples 6-9 in Table 2 are above the maximum index according to formula (I). As the index comes closer to the recommended maximum index in these Comparison Examples (see Examples 6, 7 and 8), the content of free monomeric TDI comes closer to the critical content of 0.10 wt. %, as is illustrated in Table 2. Comparison Example 9 is also not in accordance with the invention with respect to the composition of the TDI component as it uses a TDI component that contains only 80 wt. % of the 2,4-isomer. The prepolymer of Comparison Example 9 is by far the poorest in respect of its proportion of free monomeric TDI with 0.826 wt. %.

### Examples 10 and 11

### Production of NCO Prepolymers not According to the Invention

The NCO prepolymers of Comparison Examples 10 and 11 were produced in accordance with the procedure set forth for Example 1. The process parameters for Examples 10 and 11 are set forth in Table 3.

TABLE 3
Synthesis and characterisation of TDI prepolymers not according to
the invention

| | | | Comparison Examples | |
|---|---|---|---|---|
| | | | 10 | 11 |
| Polyether type | | | Polymeg 2000 | Polymeg 1000 |
| 2,4-TDI | | [wt. %] | 99.8 | 99.8 |
| 2,6-TDI | | [wt. %] | 0.2 | 0.2 |
| OH number polyol | | [mg KOH/g] | 56 | 112 |
| (mixture) | | | | |
| Index | | [NCO/OH] | 1.70 | 1.64 |
| Indexₘₐₓ according to | | [NCO/OH] | 1.72 | 1.66 |
| formula (I) | | | | |
| Reaction temperature | | [° C.] | 80 | 80 |
| NCO | (theor.) | [wt. %] | 4.24 | 2.56 |
| value | (exp.) | [wt. %] | 3.86 | 2.50 |
| Content of | | [%] | <0.02 | <0.02 |
| free TDI | | | | |
| Viscosity | at 50° C. | [mPas] | 7340 | 5350 |

*): n.d.: not determined

The Comparison Examples of Table 3 show that, although it is entirely possible to be below the maximum index with polyether polyols that contain only primary OH groups (such as, for example, Polymeg 1000 and/or Polymeg 2000), and to thereby obtain NCO prepolymers which are also below the value of 0.1 wt. % in respect of their content of free monomeric TDI, such prepolymers are markedly poorer in terms of their viscosity (and thus, processability) than the analogous prepolymers based on polyether polyols containing primarily secondary OH groups. A direct comparison of Comparison Example 10 with Example 1, and of Comparison Example 11 with Example 5 makes the differences clear.

### Examples E1 to E8

### Production of Casting Elastomers from NCO Prepolymers According to the Invention

The production of the casting elastomers was carried out in a manner known per se by heating the prepolymers according to the invention to about 55° C. and first degassing them by application of a vacuum. The chain extender was then stirred in homogeneously in the molten state, and the reacting melt was poured into molds which were preheated to about 100° C. The molded bodies were removed from the molds after about 30 minutes and then subjected to heat treatment for about 24 hours at 110° C. The mechanical properties were then determined (see Table 4).

Synthesis and properties of casting elastomers E1 to E8
Table 4A:

| Example | | E 1 | E 2 | E 3 | E 4 | E 5 | E 6 | E 7 | E 8 |
|---|---|---|---|---|---|---|---|---|---|
| Formulation | | | | | | | | | |
| Prepolymer of Ex. 1 | [pt. by wt.] | | | | | 100 | 100 | 100 | 100 |
| Prepolymer of Ex. 5 | [pt. by wt.] | 100 | 100 | 100 | 100 | | | | |
| Aromatic Amine 3 | [pt. by wt.] | 17.24 | | | | 10.39 | | | |
| Aromatic Amine 1 | [pt. by wt.] | | 10.55 | | | | 6.35 | | |
| Aromatic Amine 2 | [pt. by wt.] | | | 9.8 | | | | 5.89 | |
| Aromatic Amine 4 | [pt. by wt.] | | | | 8.11 | | | | 4.88 |
| Tinuvin ® B75 | [pt. by wt.] | | | | | 0.5 | | | 0.5 |
| Oleic acid | [pt. by wt.] | | 0.5 | | | | 0.5 | | |
| Processing | | | | | | | | | |
| Index | | 1.05 | 1.10 | 1.05 | 1.05 | 1.05 | 1.10 | 1.05 | 1.05 |
| NCO value | [wt. %] | 4.02 | 4.02 | 4.02 | 4.02 | 2.42 | 2.42 | 2.42 | 2.42 |
| Viscosity 70° C. | [mPas] | 1096 | 1096 | 1096 | 1096 | 847 | 847 | 847 | 847 |
| Prepolymer | [° C.] | 60 | 60 | 60 | 60 | 50 | 50 | 50 | 50 |
| temperature | | | | | | | | | |
| Crosslinker | [° C.] | 110 | 90 | 23 | 23 | 110 | 90 | 23 | 23 |
| temperature | | | | | | | | | |
| Casting time | [sec] | 240 | 150 | 240 | 40 | 450 | 600 | 600 | 60 |
| After-heating | [° C.] | 110 | 110 | 110 | 110 | 110 | 110 | 110 | 110 |
| temperature | | | | | | | | | |
| After-heating time | [hrs] | 24 | 24 | 24 | 24 | 24 | 24 | 24 | 24 |

Table 4B:

| Mechanical | | | | | | | | | | |
|---|---|---|---|---|---|---|---|---|---|---|
| properties | | Example | | | | | | | | |
| | DIN | | E 1 | E 2 | E 3 | E 4 | E 5 | E 6 | E 7 | E 8 |
| Hardness | 53505 | [Shore A] | 93 | 92 | 83 | 87 | 79 | 79 | 63 | 73 |
| Hardness | 53505 | [Shore D] | 39 | 35 | 26 | 30 | 25 | 25 | 16 | 21 |
| Stress | 53504 | [MPa] | 8.61 | 6.86 | 4.3 | 4.62 | 3.59 | 3.38 | 1.92 | 2.25 |
| 50% | | | | | | | | | | |
| Stress | 53504 | [MPa] | 9.6 | 7.8 | 5.5 | 5.5 | 5.0 | 4.3 | 2.6 | 2.9 |
| 100% | | | | | | | | | | |
| Stress | 53504 | [MPa] | 12.8 | 8.8 | 7.3 | 7.6 | 5.8 | 5.5 | 3.9 | 3.8 |
| 300% | | | | | | | | | | |
| Stress at | 53504 | [MPa] | 28.9 | 24.2 | 16.8 | 23.1 | 14.9 | 7.9 | 9.3 | 10.5 |
| break | | | | | | | | | | |
| Elongation | 53504 | [%] | 462 | 622 | 655 | 561 | 878 | 556 | 931 | 913 |
| at break | | | | | | | | | | |
| Graves | 53515 | [kN/m] | 42.77 | 45.7 | 45.06 | 34.46 | 25 | 20 | 17 | 23 |
| Impact | 53512 | [%] | 43 | 44 | 39 | 49 | 65 | 70 | 61 | 67 |
| resilience | | | | | | | | | | |
| Abrasion | 53516 | [mm³] | 63 | 77 | 124 | 80 | 98 | 104 | 168 | 111 |
| Density | 53420 | [g/mm³] | 1.108 | 1.109 | 1.108 | 1.089 | 1.065 | 1.062 | 1.063 | 1.051 |
| DVR 22° C. | 53517 | [%] | 20 | 31 | 35 | 44 | 16 | 13 | 33 | 50 |
| DVR 70° C. | 53517 | [%] | 47 | 66 | 78 | 72 | 41 | 34 | 77 | 89 |

Tables 4A and 4B show that the NCO prepolymers according to the invention can be processed without problems (i.e. at a low viscosity of the prepolymer, with an adequate casting time and processing temperature) with aromatic amine chain extenders to form casting elastomers, with it being possible to obtain elastomers in the hardness range of approximately from 60 to 95 Shore A, depending on the chosen prepolymer and chain extender. The other mechanical properties also vary accordingly. Hard systems have the high values known of other casting elastomers such as, for example, stress at tear. On the other hand, softer types have high values with respect to, for example, elongation at tear. Overall, the whole of the property profile specified for PUR casting elastomers on the market can be covered well with casting elastomers based on the TDI prepolymers according to the invention.

Even in the case of chain extenders such as, for example, DETDA, which normally cannot be processed by the manual casting process, it is possible to use them in the casting process. This is due to the low proportion of free monomeric TDI present in the NCO prepolymers according to the invention.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the production of NCO prepolymers based on toluene diisocyanate having a content of free monomeric toluene diisocyanate of not more than 0.1 wt. % and a viscosity, measured at 50° C., of not more than 5000 mPas, comprising
(1) reacting in a single-step at reaction temperatures of from 40 to 95° C.
(a) one or more polyether polyols having at least 85% secondary OH groups,
with
(b) toluene diisocyanate having a proportion of 2,4-isomer of more than 99.5 wt. %,
wherein the molar ratio of isocyanate groups to hydroxyl groups (or index) in the range of from 1.52:1 to 1.85:1 is maintained, with the proviso that the index does not exceed the value defined by formula (I):
indexₘₐₓ=1.5982+7/hydroxyl number of the polyether

2. An NCO prepolymer based on toluene diisocyanate having a content of free monomeric toluene diisocyanate of not more than 0.1 wt. % and a viscosity, measured at 50° C., of not more than 5000 mPas, comprising the reaction product of:
(a) one or more polyether polyols having at least 85% secondary OH groups, with
(b) toluene diisocyanate having a proportion of 2,4-isomer of more than 99.5 wt. %,
in a single-step reaction at reaction temperatures of from 40 to 95° C., wherein the molar ratio of isocyanate groups to hydroxyl groups (index) is in the range of from 1.52:1 to 1.85:1, with the proviso that the index does not exceed the value defined by formula (I):
indexₘₐₓ=1.5982+7/hydroxyl number of the polyether (I),

3. A process for the production of polyurethane east elastomer comprising reacting (1) one or more NCO prepolymers of claim 2, with (2) one or more aromatic amine chain extenders.

4. The process of claim 3, wherein (2) said aromatic amine chain extenders are selected from the group consisting of 3,5-diamino-4-chlorobenzoic acid isobutyl ester, 3,5-bis(methylthio)-2,4-diaminotoluene, 4,4′-diamino-2,2′-dichloro-5,5′-diethyldiphenylmethane, 2,4-diamino-3,5-diethyltoluene, isomers of 2,4-diamino-3,5-diethyltoluene and mixtures thereof.

5. A process for the production of foamed or non-foamed polyurethanes comprising reacting (1) one or more NCO prepolymers of claim 2, with (2) an isocyanate-reactive component.

6. A cast elastomer comprising the reaction product of (1) one or more NCO prepolymers of claim 2, with (2) one or more aromatic amine chain extenders.

7. A foamed or non-foamed polyurethane comprising the reaction product of (1) one or more NCO prepolymers of claim 2, with (2) an isocyanate-reactive component.

8. A moisture-cured film comprising the NCO prepolymers of claim 2.
